(19) United States
(12) Patent Application Publication
(10) Pub. No.: US 2008/0188657 A1
(43) Pub. Date: Aug. 7, 2008

(54) CHEMICAL PROCESS

(75) Inventor: Steven Robert LENGER, Bristol (GB)

Correspondence Address: MORGAN LEWIS & BOCKIUS LLP, 1111 PENNSYLVANIA AVENUE NW, WASHINGTON, DC, 20004, US

(73) Assignee: AstraZeneca UK Limited, London (GB)

(21) Appl. No.: 11/948,615

(22) Filed: Nov. 30, 2007

Related U.S. Application Data
(60) Provisional application No. 60/868,111, filed on Dec. 1, 2006.

(51) Int. Cl. C07D 239/42

(52) U.S. Cl. 544/297

(57) ABSTRACT

A process for the manufacture of a compound of formula (V), useful for making rosuvastatin, by a stereoselective aldol reaction is described.

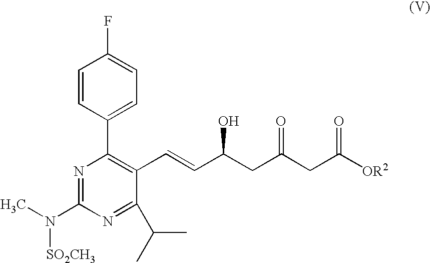

This application claims the benefit under 35 U.S.C. § 119(e) of Application No. 60/868,111 (US) filed on Dec. 1, 2006.

This invention concerns a novel chemical process, and more particularly it concerns a novel chemical process for the manufacture of rosuvastatin and its pharmaceutically acceptable salts, especially rosuvastatin calcium.

Rosuvastatin and its pharmaceutically acceptable salts are HMG CoA reductase inhibitors and have use in the treatment of, inter alia, hypercholesterolemia and mixed dyslipidemia. Rosuvastatin calcium (Formula (A)) is marketed under the trademark CRESTOR™. European Patent Application, Publication No. (EPA) 0521471 discloses (E)-7-[4-(4-fluorophenyl)-6-isopropyl-2-[methyl(methylsulfonyl)amino]pyrimidin-5-yl](3R,5S)-3,5-dihydroxyhept-6-enoic acid (rosuvastatin) and its sodium salt and calcium salt (rosuvastatin calcium, illustrated below) and a process for their preparation.

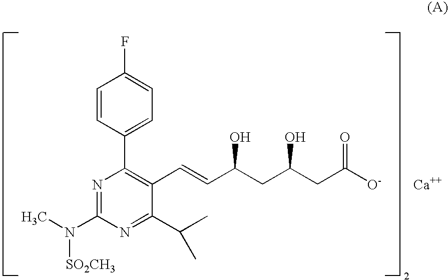

Rosuvastatin and its pharmaceutically acceptable salts are obtained therein by condensation of methyl (3R)-3-[(tert-butyldimethylsilyl)oxy]-5-oxo-6-triphenylphosphoranylidene hexanoate with 4-(4-fluorophenyl)-6-isopropyl-2-(N-methyl-N-methanesulfonylamino)-5-pyrimidinecarboxaldehyde, followed by deprotection of the 3-hydroxy group, asymmetric reduction of the 5-oxo group and hydrolysis.

Other processes for the preparation of rosuvastatin and its pharmaceutically acceptable salts are described in WO 00/49014 and WO 04/52867. The compound and its pharmaceutically acceptable salts are obtained in WO 00/49014 by reaction of diphenyl [4-(4-fluorophenyl)-6-isopropyl-2-[methyl(methylsulfonyl)amino]pyrimidin-5-ylmethyl]phosphine oxide with tert-butyl 2-[(4R,6S)-6-formyl-2,2-dimethyl-1,3-dioxan-4-yl}acetate in the presence of a base, followed by removal of protecting groups. WO 04/52867 discloses the condensation of 1-cyano-(2S)-2-[(tert-butyldimethylsilyl)oxy-4-oxo-5-triphenylphosphoranylidene pentane with 4-(4-fluorophenyl)-6-isopropyl-2-(N-methyl-N-methanesulfonylamino)-5-pyrimidinecarboxaldehyde, followed by deprotection, asymmetric reduction of the 4-oxo group and hydrolysis.

However there is a continuing need to identify alternative processes for the manufacture of rosuvastatin and its pharmaceutically acceptable salts. Such processes may, for example, when compared to previously known processes, be more convenient to use, be more suitable for large scale manufacture, give the product in a better yield, reduce the number of steps involved, use intermediates which are more easily isolated, require less complex purification techniques, use less expensive reagents and/or be more environmentally friendly.

WO 03/064382 describes a process for manufacture of statin compounds such as, inter alia, pitavastatin and rosuvastatin, based on an asymmetric aldol reaction using a chiral titanium catalyst. WO 03/42180 describes a similar process for the synthesis of pitavastatin. Our co-pending application WO2007/007119 (PCT/GB2006/003543) describes an asymmetric aldol approach to rosuvastatin, using a dienyl silylenol ether as a masked acetoacetate component in the presence of an amine such as TMEDA.

We have now discovered that an alternative diene component can be used to obtain rosuvastatin and its pharmaceutically acceptable salts in good yield and enantiomeric purity, without the need for an amine such as TMEDA.

According to a first aspect of the invention, there is provided a process for the manufacture of a compound of formula (I)

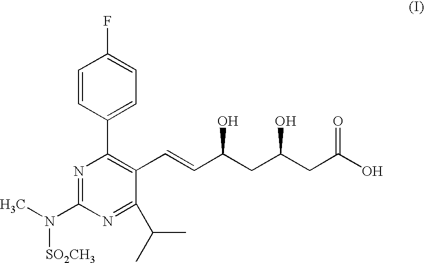

or a pharmaceutically acceptable salt thereof, comprising
a) reaction of a compound of formula (II)

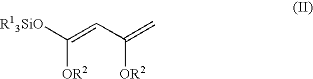

wherein each R¹ is independently selected from (1-6C)alkyl and phenyl;
each R² is independently selected from (1-6C)alkyl and aryl(1-6C)alkyl;
or the two R² groups together comprise a (1-3C) alkylene chain or (5-6C)spirocycloalkyl group (optionally substituted with 1 or 2 (1-4C)alkyl groups);
with a compound of formula (III)

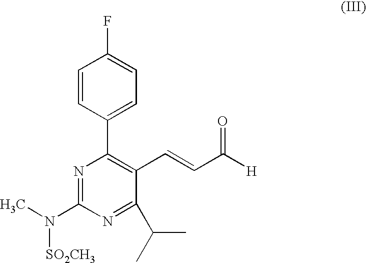

in the presence of a titanium (IV) catalyst of formula (IV)

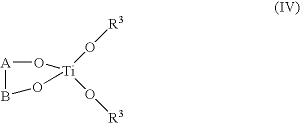

(wherein each R³ is independently selected from (1-6C)alkyl and A-B comprises an optionally substituted biaryl derivative in the S-configuration) and an alkali metal halide salt, in an inert solvent, to give a compound of formula (V);

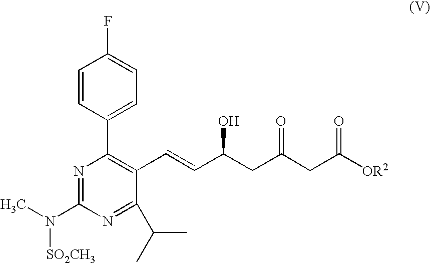

b) reduction of the keto-group in the compound of formula (V) to give a compound of formula (VI);

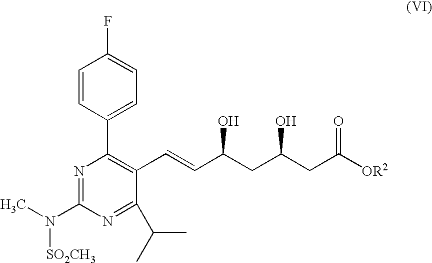

and
c) removal of the R² group to give the compound of formula (I) or a salt thereof;
optionally followed by formation of a pharmaceutically-acceptable salt.

It will be understood that using the opposite enantiomer of the titanium catalyst will give the opposite enantiomer of the compound of formula (V) and is thereby a route to make the enantiomer of rosuvastatin.

It will be understood that compounds of formula (II) wherein the two R² groups together comprise a (1-3C) alkylene chain or (5-6C)spirocycloalkyl group (optionally substituted with 1 or 2 (1-4C)alkyl groups) include the following structures:

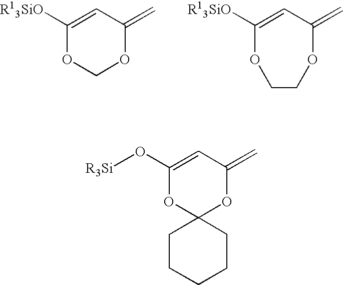

Suitable conditions for the reactions are described below.

Suitable optionally substituted biaryldioxy derivatives represented by

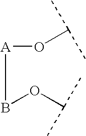

include those derived from the following diols:

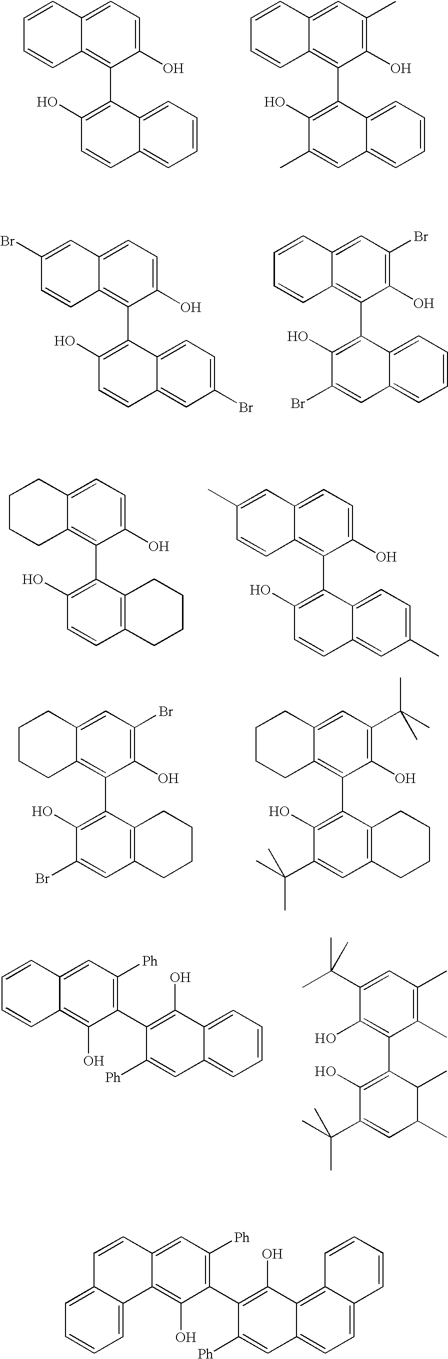

A particularly suitable biaryl derivative is that derived from the following diol:

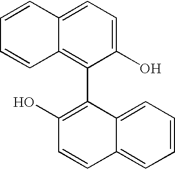

It will be understood that the above biaryl systems are chiral and are used in the S-configuration in the reaction of the invention.

### Step a)

The use of the alkali metal halide is believed to be essential for obtaining good yield and enantiomeric excess for this reaction with the compound of formula (III).

The molar ratio of the aldehyde of formula (III) and a compound of formula (II) initially present in the reaction mixtures is conveniently between 1:1 and 1:6, such as from 1:1 to 1:4, conveniently between 1:1.5 and 1:3, such as 1:2.

The molar ratio of the titanium (IV) catalyst of formula (IV) to the aldehyde of formula (III) initially present in the reaction mixture is conveniently between 0.01:1 and 0.5:1, such as between 0.1:1 and 0.3:1.

The molar ratio of the alkali metal halide to the aldehyde of formula (III) initially present in the reaction mixtures is conveniently between 0.03:1 to 1:1, particularly between 0.1:1 and 0.5:1. The exact quantity of alkali metal halide to be used will be understood by the skilled person to depend on the amount of the titanium catalyst used, and/or the concentration of the reaction solution. The quantities given above are particularly suitable when the alkali metal halide is lithium chloride.

The reaction may be carried out in a polar aprotic solvent, such as tetrahydrofuran, diethylether or dimethoxyethane, preferably tetrahydrofuran. A combination of solvents may also be used.

The reaction may be carried out at a temperature from about 0° C. to about 70° C., such as from about 10° C. to about 60° C. and preferably from about 15° C. to about 30° C.

A preferred alkali metal halide is lithium chloride.

Examples of (1-6C)alkyl and (1-4C)alkyl include methyl, ethyl, propyl, isopropyl and tert-butyl. Examples of aryl(1-6C)alkyl include benzyl. Examples of (1-3C)alkylene include methylene, ethylene and propylene. Examples of (5-6C)spiroalkyl include spirocyclopentyl and spirocyclohexyl. Examples of (3-6C)cycloalkyl include cyclopropyl, cyclobutyl, cyclopentyl and cyclohexyl.

Suitably each R¹ group is methyl. Suitably each R² is independently selected from (1-6C)alkyl, particularly each R² is ethyl.

A compound of formula (II) may be prepared according to the procedures described in Organic Letters, 2005, 7, 2421-2423. Spirocyclic compounds of formula (II) may be prepared by methods known in the art. Suitable starting materials for such compounds include spirocyclic acetoacetates such as:

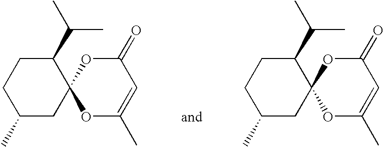

A compound of formula (IV) may be prepared according to the procedures described in WO03/064382 and WO03/42180.

A compound of formula (III) may be made by the following procedure, as illustrated in the accompanying Examples and as shown in Scheme 1 below.

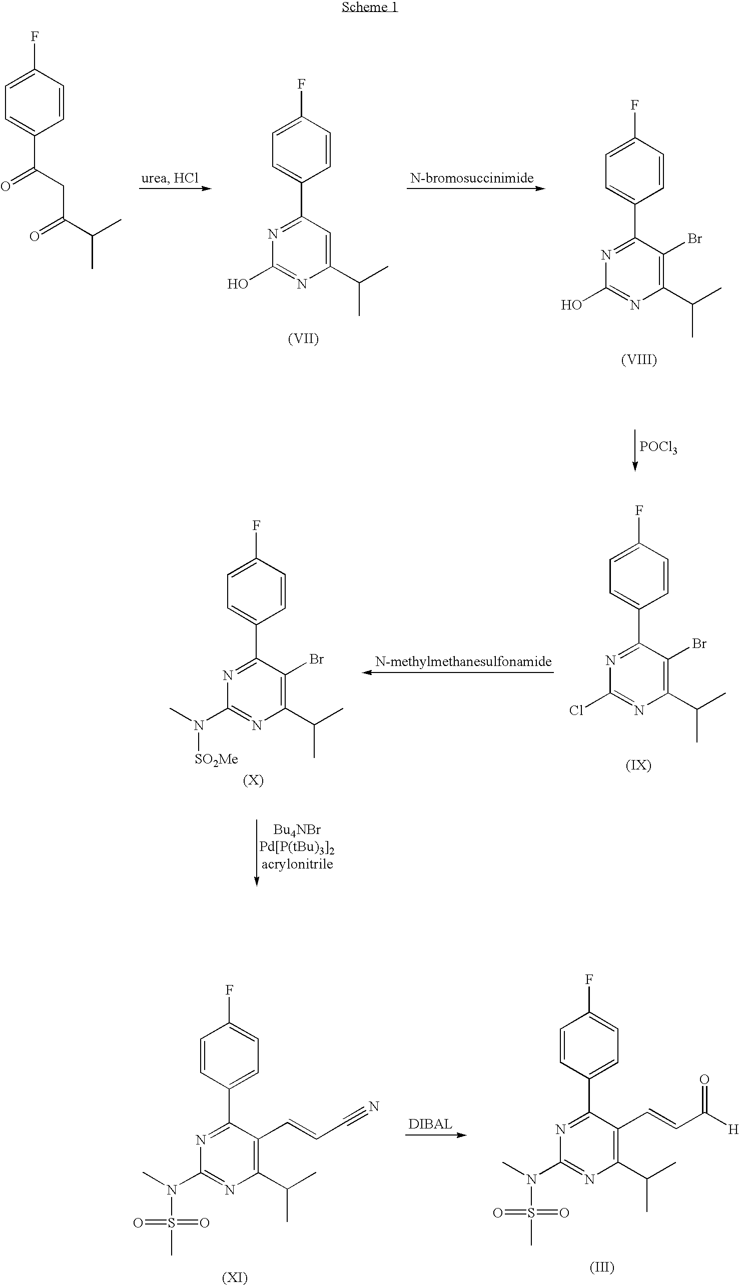

It will be understood that the present invention encompasses the use of the compound of formula (III) made by any suitable method and is not restricted to that shown in the above scheme.

Suitably the compound of formula (XI) may be made by reacting the compound of formula (X) with acrylonitrile in the presence of a transition metal catalyst, such as a palladium catalyst, such as Pd[P(tBu)₃]₂ [pre-prepared or generated in situ from, for example bis(dibenzylideneacetone)palladium(0) (Pd(dba)₂) or tris(dibenzylideneacetone)dipalladium(0) (Pd₂(dba)₃) and ^{t}Bu₃PH·BF₄]. A phase transfer catalyst, such as tetrabutylammonium bromide may be used.

Suitably, conversion of the compound of formula (XI) to the compound of formula (III) may be carried out by reduction using DIBAL (diisobutylaluminium hydride). Further suitable reducing agents include the following and complexes thereof: Raney nickel (with a source of H₂), tin(II) chloride, lithium triethylborohydride, potassium 9-sec-amyl-9-boratabicyclo[3.3.1]nonane, diisopropylaluminum hydride, lithium triethoxyaluminum hydride, lithium diethoxyaluminum hydride, sodium diethylaluminum hydride, lithium aluminium hydride, lithium tris(dialkylamino)aluminium hydrides, and trialkylsilanes in the presence of appropriate Lewis acids.

More suitably, conversion of the compound of formula (XI) to the compound of formula (III) may be carried out by reduction using DIBAL, for example in toluene at <0° C.

Further suitable conditions for these reactions may be found in the accompanying examples, or are well known in the art.

An alternative process for making the compound of formula (III) is by reaction of a compound of formula (X) with an appropriate vinylic boron species such as with a vinyl boronate of formula (XII)

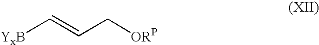

wherein BYₓ is selected from B(OH)₂, B(OH)₃⁻, B(OH)₂F⁻, BX₃⁻ (wherein X=halogen), B(OR⁵)₂, B(OR⁵)₂F, B(OR⁵)₂(OH)⁻, B(OR⁶)(OR⁷), B(OR⁶)(OR⁷)(OH)⁻, B(OR⁶)(OR⁷)F⁻, BR⁵₂, BR⁵₂OH⁻ and BR⁵F⁻;
R⁵ is selected from (1-6C)alkyl, (3-6C)cycloalkyl and aryl(1-6C)alkyl;
R⁶ and R⁷ together form a two or three carbon alkylene bridge between the two oxygens to which they are attached, optionally substituted by 1, 2, 3 or 4 methyl or phenyl groups;
or R⁶ and R⁷ together form a phenyl ring;
and R^{P} is a protecting group;
followed by deprotection to give a compound of formula (XIII):

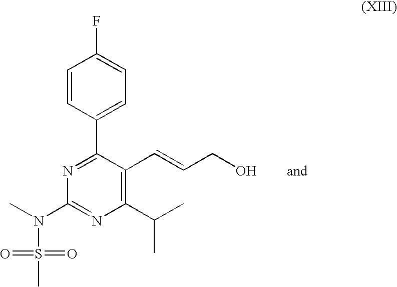

oxidation of the compound of formula (XIII) to give the compound of formula (III).

Suitable values for R^{P} include well known hydroxy protecting groups, and include for example Si(R⁴)₃ (wherein each R⁴ is independently selected from (1-6C)alkyl), tetrahydropyranyl, benzyl, p-methoxybenzyl, methoxymethyl (MOM) and benzyloxymethyl (BOM). Preferably ORP is not an ester group.

In one aspect, R^{P} is Si(R⁴)₃ (for example trimethylsilyl, or tertbutyldimethylsilyl). In another aspect R^{P} is tetrahydropyranyl.

Suitably BYₓ is B(OR⁶)(OR⁷).

Examples of B(OR⁶)(OR⁷) include:

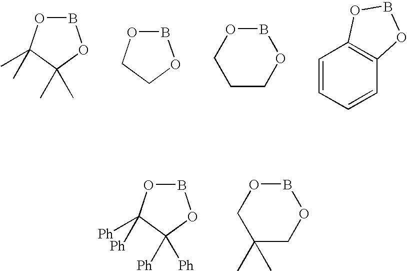

In one aspect, B(OR⁶)(OR⁷) is:

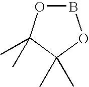

Suitably the reaction of (XII) with (X) may be carried out in the presence of a palladium catalyst such as (1,1′-bis(di-tert-butylphosphino)ferrocene)palladium(II) chloride. The reaction may be carried out in acetonitrile and water, in the presence of a base, such as potassium carbonate. Alternatively, the reaction may be carried out in the presence of fluoride, see for example J. Org. Chem., 1994, 59, 6095-6097.

It will be appreciated that for some values of R^{P} (for example when R^{P} is Si(R⁴)₃, the silyl group may be removed in situ during step A). When R^{P} is tetrahydropyranyl, a separate step may be required to deprotect the intermediate allyl ether to give the alcohol (XIII); this may be carried out for example by hydrolysis using aqueous hydrochloric acid. This deprotection step may be carried out without isolation of the intermediate allyl ether, as illustrated in the accompanying examples. When R^{P} is p-methoxybenzyl group, it may be removed under oxidative conditions which simultaneously oxidise the hydroxy group to give an aldehyde of formula (III).

Suitably the oxidation of (XIII) to give (III) (Step B) may be carried out using manganese dioxide, for example in toluene. Other oxidation conditions well known in the art may also be used, for example variations on the Swern oxidation, such as would be achieved using chlorine and dimethylsulfide.

Further suitable conditions for these reactions may be found in the accompanying examples.

It will be understood that the reaction of (II) with (III) in the presence of (IV) passes through an intermediate enolate of formula (Va), which is generally hydrolysed during work up to give compound (V). In another aspect of the invention, (Va) may be isolated and then hydrolysed to give (V) in a separate step of the reaction, for example using aqueous acid such as aqueous hydrochloric acid, for example in tetrahydrofuran. This step is referred to as step a′) hereinafter.

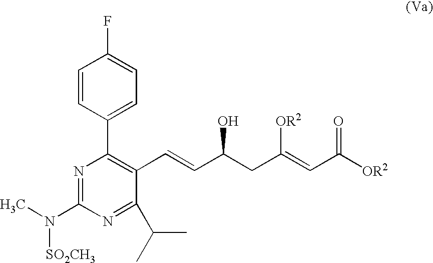

Certain compounds of formula (Va), particularly where each R² is independently selected from (1-6C)alkyl, are novel and are provided as further aspects of the invention.

When each R² is ethyl, the compound of formula (Va) is (S)-trans-ethyl 3-ethoxy-7-(4-(4-fluorophenyl)-6-isopropyl-2-(N-methylmethylsulfonamido)pyrimidin-5-yl)-5-hydroxyhept-2,6-dienoate; this compound is provided as a further aspect of the invention.

### Step b)

Reduction of the keto group in the compound of formula (V) may be carried out in the presence of a di(loweralkyl)methoxyborane, such as diethylmethoxyborane or dibutylmethoxyborane. Suitably diethylmethoxyborane is used. The reaction is generally carried out in a polar solvent, such as tetrahydrofuran or an alcohol such as methanol or ethanol, or a mixture of such solvents, for example a mixture of tetrahydrofuran and methanol.

The reducing agent is suitably a hydride reagent such as sodium or lithium borohydride, particularly sodium borohydride.

The reaction may be carried out at reduced temperatures, such as about −20° C. to about −100° C., particularly about −50° C. to about −80° C.

Similar diastereoselective reductions are described in EP0521471.

### Step c)

The R² group in the compound of formula (VI) may be removed by hydrolysis under conditions well known in the art, to form the compound of formula (I), or a salt thereof. Such salts may be pharmaceutically-acceptable salts, or may be transformed into pharmaceutically-acceptable salts. For example, R² may be hydrolysed by treatment with aqueous sodium hydroxide to form the sodium salt of (I).

A suitable pharmaceutically acceptable salt includes, for example, an alkali metal salt, for example a sodium or potassium salt, an alkaline earth metal salt, for example, calcium or magnesium salt, an ammonium salt or a salt with an organic base which affords a physiologically-acceptable cation, for example with methylamine, ethylamine, dimethylamine, trimethylamine, morpholine, diethanolamine, tris(2-hydroxyethyl)amine and tris(hydroxymethyl)methylamine.

The compound of formula (I) is marketed as its calcium salt as described hereinbefore. The calcium salt may be formed directly as a product of the reaction to remove the R² group (for example by treating the compound of formula (VI) with aqueous calcium hydroxide, see patent application US 2003/0114685) or by treating an alternative salt of the compound of formula (I), such as the sodium salt, with an aqueous solution of a suitable calcium source. Suitable calcium sources include calcium chloride and calcium acetate. This is illustrated in Scheme 2:

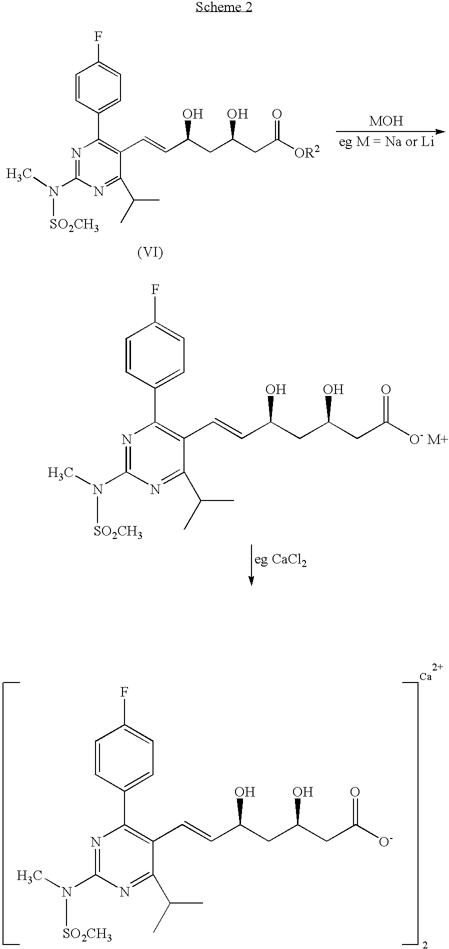

Suitable conditions for transformation of the sodium salt to the calcium salt are described in EP0521471. It will be appreciated that the resulting calcium salt may be retreated if desired in order to obtain different particle size, or different physical form (such as amorphous vs crystalline) by processes known in the art (see for example International Patent Applications WO00/42024 and WO2005/023779).

According to another aspect of the invention, there is provided a process for the manufacture of a compound of formula (I)

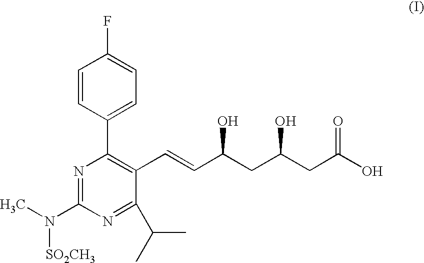

or a pharmaceutically acceptable salt thereof, comprising
a) reaction of a compound of formula (II)

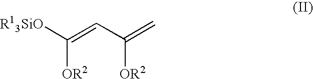

wherein each R¹ is independently selected from (1-6C)alkyl and phenyl;
each R² is independently selected from (1-6C)alkyl and aryl(1-6C)alkyl; or the two R² groups together comprise a (1-3C) alkylene chain or (5-6C)spirocycloalkyl group (optionally substituted with 1 or 2 (1-4C)alkyl groups);
with a compound of formula (III)

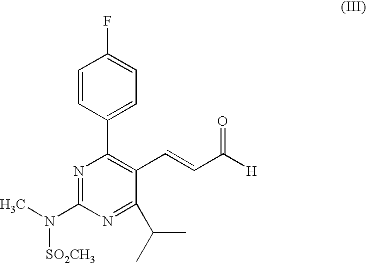

in the presence of a titanium (IV) catalyst of formula (IV)

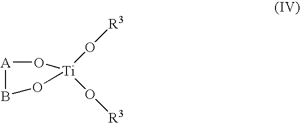

(wherein each R³ is independently selected from (1-6C)alkyl and A-B comprises an optionally substituted biaryl derivative in the S-configuration) and an alkali metal halide salt, in an inert solvent, to give a compound of formula (Va);

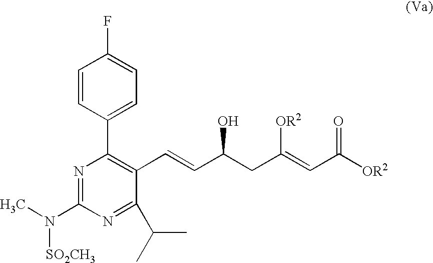

a′) hydrolysis of (Va) to give a compound of formula (V);

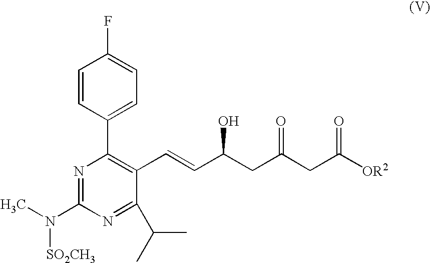

b) reduction of the keto-group in the compound of formula (V) to give a compound of formula (VI);

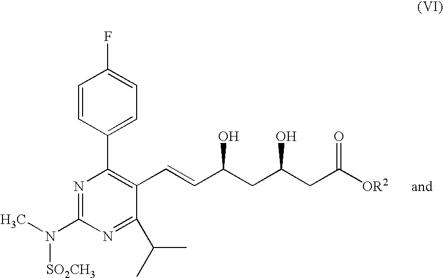

c) removal of the R² group to give the compound of formula (I) or a salt thereof;
optionally followed by formation of a pharmaceutically-acceptable salt.

In a further aspect of the invention, there is provided a process for the manufacture of a compound of formula (VI)

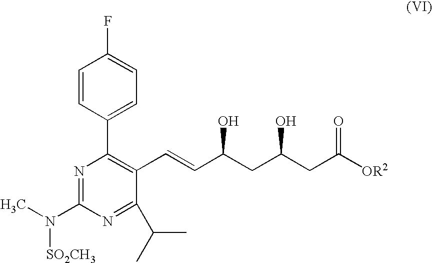

comprising:
a) reaction of a compound of formula (II)

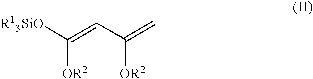

wherein each R¹ is independently selected from (1-6C)alkyl and phenyl;
each R² is independently selected from (1-6C)alkyl and aryl(1-6C)alkyl; or the two R² groups together comprise a (1-3C) alkylene chain or (5-6C)spirocycloalkyl group (optionally substituted with 1 or 2 (1-4C)alkyl groups); with a compound of formula (III)

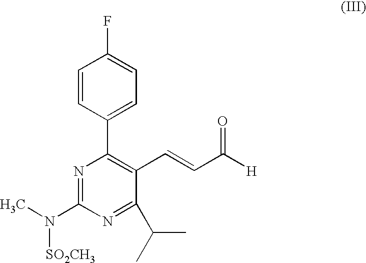

in the presence of a titanium (IV) catalyst of formula (IV)

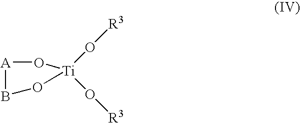

(wherein each R³ is independently selected from (1-6C)alkyl and A-B comprises an optionally substituted biaryl derivative in the S-configuration), an alkali metal halide salt in an inert solvent, to give a compound of formula (V);

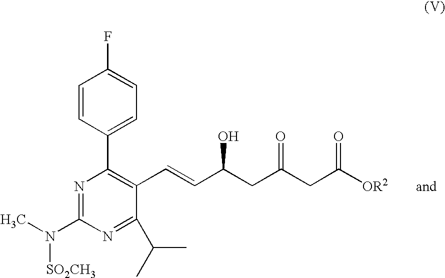

b) reduction of the keto-group in the compound of formula (V) to give a compound of formula (VI).

In a further aspect of the invention, there is provided a process for the manufacture of a compound of formula (VI)

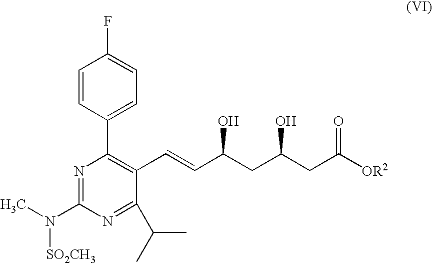

comprising:
a) reaction of a compound of formula (II)

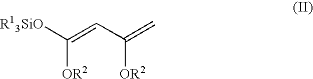

wherein each R¹ is independently selected from (1-6C)alkyl and phenyl;
each R² is independently selected from (1-6C)alkyl and aryl(1-6C)alkyl;
or the two R² groups together comprise a (1-3C) alkylene chain or (5-6C)spirocycloalkyl group (optionally substituted with 1 or 2 (1-4C)alkyl groups);
with a compound of formula (III)

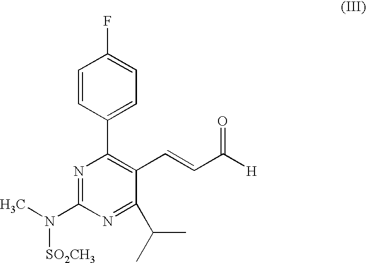

in the presence of a titanium (IV) catalyst of formula (IV)

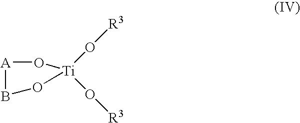

(wherein each R³ is independently selected from (1-6C)alkyl and A-B comprises an optionally substituted biaryl derivative in the S-configuration) and an alkali metal halide salt, in an inert solvent, to give a compound of formula (Va);

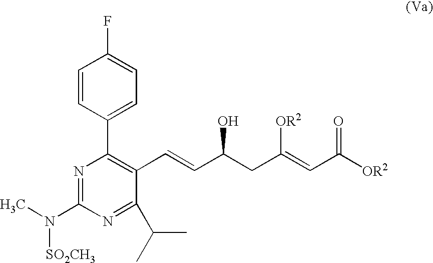

a′) hydrolysis of (Va) to give a compound of formula (V);

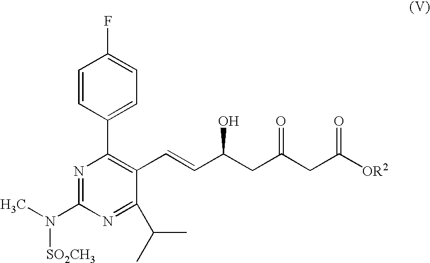

b) reduction of the keto-group in the compound of formula (V) to give a compound of formula (VI).

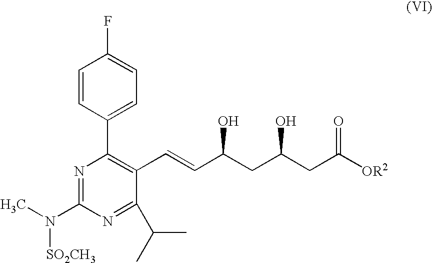

Suitable conditions for steps a), a′) and b) are as hereinbefore described.

In a further aspect of the invention there is provided a process for the manufacture of a compound of formula (V)

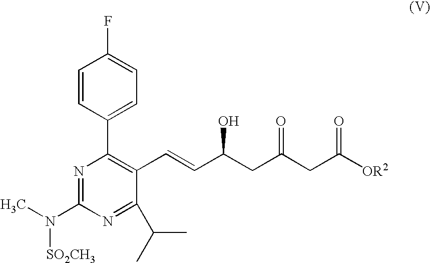

comprising
reaction of a compound of formula (II)

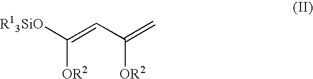

wherein each R¹ is independently selected from (1-6C)alkyl and phenyl;
each R² is independently selected from (1-6C)alkyl and aryl(1-6C)alkyl;
or the two R² groups together comprise a (1-3C) alkylene chain or (5-6C)spirocycloalkyl group (optionally substituted with 1 or 2 (1-4C)alkyl groups);
with a compound of formula (III)

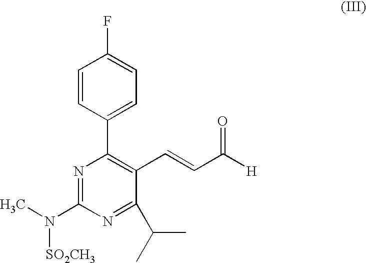

in the presence of a titanium (IV) catalyst of formula (IV)

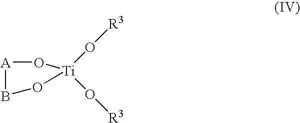

(wherein each R³ is independently selected from (1-6C)alkyl and A-B comprises an optionally substituted biaryl derivative in the S-configuration) and an alkali metal halide salt in an inert solvent.

In a further aspect of the invention there is provided a process for the manufacture of a compound of formula (V)

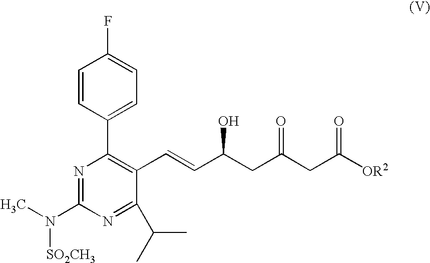

comprising
reaction of a compound of formula (II)

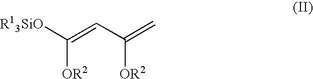

wherein each R¹ is independently selected from (1-6C)alkyl and phenyl;
each R² is independently selected from (1-6C)alkyl and aryl(1-6C)alkyl;
or the two R² groups together comprise a (1-3C) alkylene chain or (5-6C)spirocycloalkyl group (optionally substituted with 1 or 2 (1-4C)alkyl groups);
with a compound of formula (III)

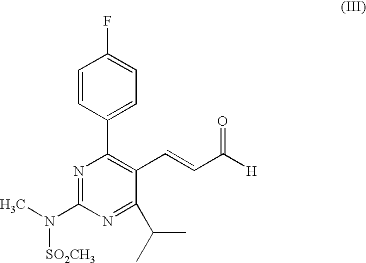

in the presence of a titanium (IV) catalyst of formula (IV)

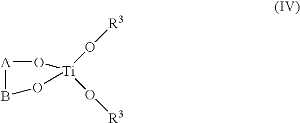

(wherein each R³ is independently selected from (1-6C)alkyl and A-B comprises an optionally substituted biaryl derivative in the S-configuration) and an alkali metal halide salt in an inert solvent to give a compound of formula (Va);

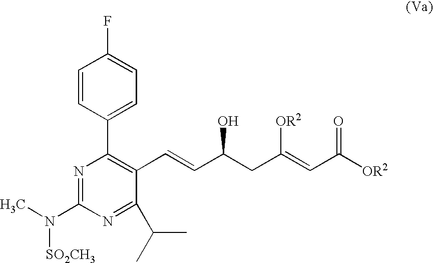

a′) hydrolysis of (Va) to give a compound of formula (V).

Suitable conditions for this reaction are as described hereinbefore for process a) and a′).

In a further aspect of the invention there is provided a process for the manufacture of a compound of formula (VI) comprising

a) forming a compound of formula (V) as hereinbefore described; and further comprising
b) reduction of the keto-group in the compound of formula (V) to give a compound of formula (VI).

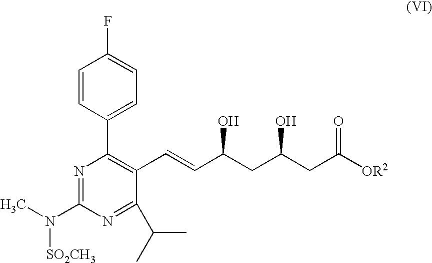

According to a further aspect of the invention, there is provided a process for forming a compound of formula (I) or a pharmaceutically acceptable salt thereof, comprising

a) forming a compound of formula (V) and b) forming a compound of formula (VI) as hereinbefore described; and further comprising
c) removal of the R² group to give the compound of formula (I) or a salt thereof;
optionally followed by formation of a pharmaceutically-acceptable salt.

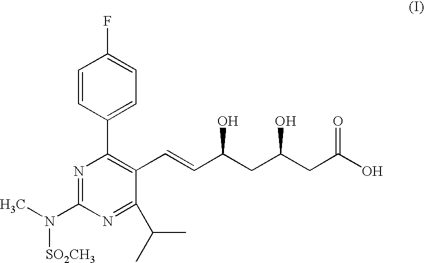

Under certain conditions, as illustrated in the accompanying examples, it is possible to carry out the reduction of compound (V) to compound (VI) and the subsequent conversion to compound (I) or a salt thereof, without isolation of the intermediate compound (VI). Telescoping two reactions into one step in this way would be expected to be efficient and cost effective, provided product quality is not compromised.

According to a further aspect of the invention, there is provided a process for formation of a compound of formula (I) or a salt thereof, wherein steps b) and c) are carried out without isolation of the intermediate compound of formula (VI).

### EXAMPLES

In the following non-limiting Examples, unless otherwise stated:
(i) evaporations were carried out by rotary evaporation in vacuo and work-up procedures were carried out after removal of residual solids such as drying agents by filtration;
(ii) operations were carried out at room temperature, that is in the range 18-25° C. and under an atmosphere of an inert gas such as argon or nitrogen;
(iii) yields are given for illustration only and are not necessarily the maximum attainable;
(iv) the structures of the end-products were confirmed by nuclear (generally proton) magnetic resonance (NMR); proton magnetic resonance chemical shift values were measured on the delta scale (relative to tetramethylsilane) and peak multiplicities are shown as follows: s, singlet; d, doublet; t, triplet; m, multiplet; br, broad; q, quartet, quin, quintet;
(v) intermediates were not necessarily fully characterised and purity was assessed by thin layer chromatography (TLC), melting point (Mp), high-performance liquid chromatography (HPLC), infra-red (IR) or NMR analysis;
(vi) Purification by chromatography generally refers to flash column chromatography, on silica unless otherwise stated. Column chromatography was generally carried out using prepacked silica cartridges (from 4g up to 400 g) such as Biotage (Biotage UK Ltd, Hertford, Herts, UK), eluted using a pump and fraction collector system.
(vii) High Resolution Mass spectra (HRMS) data was generated using a Micromass LCT time of flight mass spectrometer.
(viii) melting point data were generally measured using Differential Scanning Calorimetry (DSC) using a Perkin Elmer Pyris 1. Values quoted are onset temperature.

The invention will be illustrated by the following examples, in which the following abbreviations are used:

DIBAL di-isobutyl aluminium hydride

DCM dichloromethane

EtOAc ethylacetate

CDCl₃ deuterochloroform

DMF dimethylformamide

MTBE methyl tert-butyl ether

e.e. enantiomeric excess

### Example 1

### (3R,5S)-trans-7-(4-(4-fluorophenyl)-6-isopropyl-2-(N-methylmethylsulfonamido)pyrimidin-5-yl)-3,5-dihydroxyhept-6-enoic acid, calcium salt

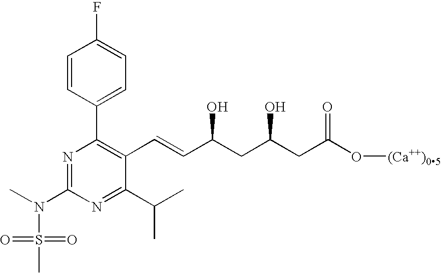

Under a nitrogen atmosphere, (S)-trans-ethyl 7-(4-(4-fluorophenyl)-6-isopropyl-2-(N-methylmethylsulfonamido)pyrimidin-5-yl)-5-hydroxy-3-oxohept-6-enoate (200 mg, 0.39 mmol, 99.3% e.e.) and methanol (0.67 mL) were dissolved in 5 mL tetrahydrofuran and cooled to −70° C. To this solution was added diethylmethoxyborane (1 M in tetrahydrofuran, 430 μL, 0.43 mmol) dropwise via syringe over 25 minutes. The resulting pale yellow solution was stirred 30 minutes at −78° C., then sodium borohydride (16.3 mg, 0.43 mmol) was added. The mixture was stirred for two hours at −78°, then the reaction was quenched with acetic acid (86 mg, 1.44 mmol) and allowed to warm to room temperature. To this was added 2 mL of 1M aqueous NaOH, and the resulting solution was stirred for 90 minutes. This was then diluted with 5 mL water and 5 mL toluene, stirred 30 minutes, separated, and aqueous concentrated in vacuo to give a pale oil. The oil was dissolved in 5 mL water, heated to 40° C., then aqueous calcium chloride (0.93 M, 300 μL, 0.28 mmol) was added dropwise via syringe. The resulting slurry was cooled to room temperature over 60 minutes, then the solids were collected via filtration with a 1 mL water wash. The collected solids were dried overnight under vacuum to yield (3R,5S)-trans-7-(4-(4-fluorophenyl)-6-isopropyl-2-(N-methylmethylsulfonamido)pyrimidin-5-yl)-3,5-dihydroxyhept-6-enoic acid, calcium salt (122.6 g, 62% yield, 99.3% e.e.) as a white crystalline solid. Physical data were identical to existing standard and its published description.

### 1a): (3R,5S)-trans-ethyl 7-(4-(4-fluorophenyl)-6-isopropyl-2-(N-methylmethylsulfonamido)pyrimidin-5-yl)-3,5-dihydroxyhept-6-enoate

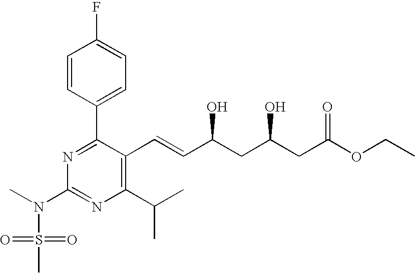

Under a nitrogen atmosphere, (S)-trans-ethyl 7-(4-(4-fluorophenyl)-6-isopropyl-2-(N-methylmethylsulfonamido)pyrimidin-5-yl)-5-hydroxy-3-oxohept-6-enoate (506 mg, 1.00 mmol) and methanol (1.7 mL) were dissolved in 10 mL tetrahydrofuran and cooled to −76° C. To this solution was added diethylmethoxyborane (1.0 M in tetrahydrofuran, 1.15 mL, 1.15 mmol) dropwise via syringe over 30 minutes. The resulting pale yellow solution was stirred 30 minutes at −75° C., then sodium borohydride (43.5 mg, 1.15 mmol) was added. The reaction was stirred for two hours at −65° C., then the reaction was quenched with acetic acid (224 μL, 3.75 mmol) and allowed to warm to room temperature. It was diluted with 100 mL of methyl tert-butyl ether and 20 mL water, stirred vigorously for 10 minutes, then separated. The upper organic phase was washed with 20 mL water, 20 mL saturated aqueous NaHCO₃ solution, and then with 20 mL water, then concentrated in vacuo to give a pale oil, which was purified by Biotage chromatography (50:50 EtOAc/hexane) to yield the title product (182 mg, 36% yield) as a white solid. ¹H NMR (400 MHz) (CDCl₃) δ: 1.27 (6H, d), 1.28 (3H, t), 2.45 (1H, s), 2.47 (1H, d), 3.37 (1H, m), 3.52 (3H, s), 3.57 (3H, s), 3.58 (1H, br. s), 3.74 (1H, br. s.), 4.19 (2H, q), 4.22 (1H, m), 4.46 (1H, m), 5.46 (1H, dd), 6.64 (1H, dd), 7.09 (2H, dd), 7.65 (2H, dd). Mp: 92-94° C.

HRMS calculated for C₂₄H₃₂FN₃O₆S 509.1996, found 509.1999.

### 1b): (S)-trans-Ethyl 7-(4-(4-fluorophenyl)-6-isopropyl-2-(N-methylmethylsulfonamido)pyrimidin-5-yl)-5-hydroxy-3-oxohept-6-enoate

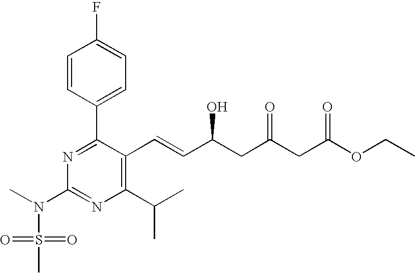

(S)-trans-Ethyl 3-ethoxy-7-(4-(4-fluorophenyl)-6-isopropyl-2-(N-methylmethylsulfonamido)pyrimidin-5-yl)-5-hydroxyhept-2,6-dienoate (130 mg, 0.101 mmol) was dissolved in tetrahydrofuran (5 mL) and cooled to 0° C. Aqueous hydrochloric acid (2.0 M, 0.75 mL, 1.50 mmol) was added via syringe and the resulting solution was warmed to room temperature, stirred for 90 minutes, then diluted with EtOAc (20 mL) and water (10 mL). The layers were separated and the organic layer washed with water (10 mL), dried with MgSO₄, and concentrated in vacuo to give the title compound (113.5 mg, 92% yield) as a pale oil in 97.46% enantiomeric excess. ¹H NMR (400 MHz) (CDCl₃) δ: 1.26 (6H, d), 1.28 (3H, t), 2.65 (1H, d), 2.66 (1H, s), 2.89 (1H, br. s), 3.34 (1H, m), 3.44 (2H, s), 3.51 (3H, s), 3.57 (3H, s), 4.21 (2H, q), 4.65 (1H, m), 5.45 (1H, dd), 6.67 (1H, dd), 7.11 (2H, dd), 7.63 (2H, dd). HRMS calculated for C₂₄H₃₀FN₃O₆S 507.1839, found 507.1870.

### 1c): (S)-trans-Ethyl 3-ethoxy-7-(4-(4-fluorophenyl)-6-isopropyl-2-(N-methylmethylsulfonamido)pyrimidin-5-yl)-5-hydroxyhept-2,6-dienoate

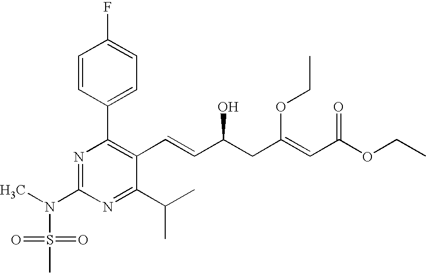

Under a nitrogen atmosphere, trans-N-(4-(4-fluorophenyl)-6-isopropyl-5-(3-oxoprop-1-enyl)pyrimidin-2-yl)-N-methylmethanesulfonamide (200 mg, 0.530 mmol), (S)-(−)-1,1′-bi-(2-naphthyloxy)(diisopropoxy)titanium (48 mg, 0.11 mmol), and lithium chloride (9.0 mg, 0.21 mmol) were dissolved in tetrahydrofuran (5 mL) at room temperature. The resulting red solution was stirred for 5 minutes, then cooled to 0° C. To this solution was added 1,3-diethoxy-1-trimethylsiloxybuta-1,3-diene (244 mg, 1.06 mmol) dropwise over 10 minutes. The resulting mixture was stirred for 42 hours at room temperature, then quenched at 0° C. with 25% aqueous formic acid (0.50 mL) and allowed to warm to room temperature. The mixture was stirred for 2 hours, then diluted with methyl tert-butyl ether (20 mL) and water (10 mL). The layers were separated and the organic layer washed with water (10 mL), dried with MgSO₄, and concentrated in vacuo to give a light yellow oil. This was purified by flash chromatography (2:1 hexane/EtOAc) to yield the title compound (145.2 mg, 54% yield) as a light yellow oil. ¹H NMR (400 MHz) (CDCl₃) δ: 1.25 (1H, t), 1.27 (6H, d), 1.32 (3H, t), 2.12 (1H, br. s), 2.29 (1H, d), 2.30 (1H, s), 3.35 (1H, m), 3.51 (3H, s), 3.57 (3H, s), 4.11 (2H, q), 4.17 (2H, ddd), 4.40 (1H, m), 4.99 (1H, s), 5.51 (1H, dd), 6.63 (1H, dd), 7.10 (2H, dd), 7.63 (2H, dd).

HRMS calculated for C₂₆H₃₄FN₃O₆S 535.2152, found 535.2215.

### 1d): (S)-(−)-1,1′-bi-(2-naphthyloxy)(diisopropoxy)titanium

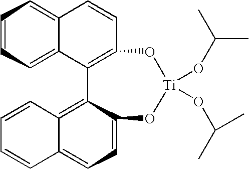

Under a nitrogen atmosphere, (S)-(−)-1,1′-bi(2-naphthol) (500 mg, 1.74 mmol), titanium tetraisopropoxide (500 μL, 1.69 mmol) and powdered 4 Å molecular sieves (500 mg) were suspended in dichloromethane (25 mL) and stirred for one hour at room temperature. The solids were filtered off, and the filtrate concentrated in vacuo to provide (S)-(−)-1,1′-bi-(2-naphthyloxy)(diisopropoxy)titanium (980 mg, 126% yield) as a dark red powder which was used in subsequent reactions without further purification.

### 1e): 4-(4-Fluorophenyl)-6-isopropylpyrimidin-2-ol

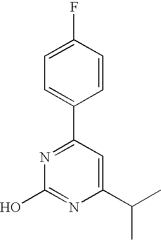

The reactor used for this experiment was thoroughly dried by carrying out a toluene distillation prior to use. Fresh toluene (100 mL) and potassium tert-butoxide (7.50 g, 64.8 mmol) were charged to the vessel and stirred to form a slurry. The mixture was cooled to −9° C. and 3-methyl-2-butanone (3.63 g, 41.7 mmol) added. The mixture was warmed to −5° C. and stirred for 30 mins. Ethyl-4-fluorobenzoate (6.25 g, 36.8 mmol) was dissolved in toluene (4 mL) and added via a syringe followed by a small toluene (1 ml) line wash. The mixture was stirred for 10 minutes at 0° C., warmed to 10° C., and then stirred at this temperature overnight. The mobile slurry was warmed to 25° C. and acetic acid (4.4 mL) added, followed by water (37.5 mL). The mixture was stirred thoroughly for 5 minutes and then allowed to stand. The lower phase was run off and discarded. A 5% sodium bicarbonate solution (16 mL) was charged to the upper phase, stirred for 5 minutes and then allowed to stand. The lower aqueous layer was run off and the upper organic phase washed twice with water (5 mL).

The remaining toluene solution was dried by azeotropic distillation (refluxing with Dean-Stark trap in place) and the solution cooled to 60° C. Urea (5.1 g, 84.9 mmol) and isopropanol (20 mL) were charged and stirred vigorously during the addition of hydrochloric acid (5 to 6 M in isopropanol, 32.3 mL, 183 mmol). The solution was heated to 80° C. and stirred for 48.5 hours before charging more hydrochloric acid in isopropanol (2 mL, 11 mmol). After a total of 112 hours at 80° C., the mixture was cooled to 60° C. and water (50 mL) added. After stirring for 15 minutes, the mixture was allowed to stand and the lower aqueous phase run off and retained. The aqueous phase was stirred and sodium hydrogen carbonate (6.9 g) added portion wise until pH=7. The product crystallised from solution and was then cooled to 20° C. The solid was filtered off and washed twice with water (20 mL) and dried in a vacuum oven at 50° C. overnight. 4-(4-fluorophenyl)-6-isopropylpyrimidin-2-ol (4.92 g) was isolated as a white powder in 56% overall yield; ¹H NMR (400 MHz; CDCl₃) δ: 1.41 (6H, d), 3.08 (1H, m), 6.69 (1H, s), 7.17 (2H, dd), 8.14 (2H, dd), 13.57 (1H, br. s). Mp: 215-217° C. HRMS calculated for C₁₃H₁₃N₂OF 232.1012, found 232.0963; used in subsequent reaction without further purification.

### 1f): 5-Bromo-4-(4-fluorophenyl)-6-isopropylpyrimidin-2-ol

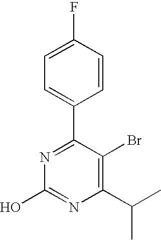

4-(4-Fluorophenyl)-6-isopropylpyrimidin-2-ol (8.00 g, 34.1 mmol) was charged to a reactor followed by DMF (100 mL). The suspension was stirred, cooled to −3° C. and N-bromosuccinimide (6.25 g, 34.8 mmol) added. The reaction mixture was warmed to 20° C. and stirred overnight. Water (100 mL) was charged to the reaction mixture and the crystalline mixture stirred for 1 hour before filtering off. The isolated solid was washed twice with water (25 mL) and the solid dried in a vacuum oven at 50° C. 5-Bromo-4-(4-fluorophenyl)-6-isopropylpyrimidin-2-ol (10.45 g, 97% yield) was obtained as a white solid;

¹H NMR (400 MHz; CDCl₃) δ: 1.39 (6H, d), 3.57 (1H, m), 7.16 (2H, dd), 7.66 (2H, dd). Mp: Decomposes at 199° C. HRMS calculated for C₁₃H₁₂N₂OFBr 310.0117, found 310.0116; used in subsequent reaction without further purification.

### 1g): 5-Bromo-2-chloro-4-(4-fluorophenyl)-6-isopropylpyrimidine

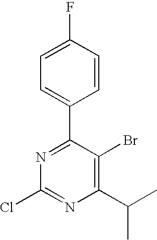

Phosphoryl chloride (5.00 mL, 53.8 mmol) was added to 5-bromo-4-(4-fluorophenyl)-6-isopropylpyrimidin-2-ol (5.027 g, 15.28 mmol) and the reaction mixture was heated to an internal temperature of 90° C. The mixture was then stirred for 150 minutes at this temperature, then allowed to cool to 25° C. The reaction mixture was quenched by dropwise addition (with 30 mL of EtOAc rinses) into a stirred mixture of ice (60 g), water (40 mL), and sodium bicarbonate (10 g). After completion of the addition, sodium bicarbonate (13 g) added to assure neutrality. The mixture was then extracted with ethyl acetate (4×70 mL). The organic phases were combined and dried with anhydrous magnesium sulphate. The solution was filtered through a pad of diatomaceous earth, and concentrated in vacuo to yield the title compound (4.98 g, 99% yield).

¹H NMR (400 MHz; CDCl₃) δ: 1.34 (6H, d), 3.64 (1H, m), 7.17 (2H, dd), 7.73 (2H, dd). Mp: 99-101° C. HRMS calculated for C₁₃H₁₁N₂FClBr 327.9778, found 327.9752; used in subsequent reaction without further purification.

### 1h): N-(5-Bromo-4-(4-fluorophenyl)-6-isopropylpyrimidin-2-yl)-N-methylmethanesulfonamide

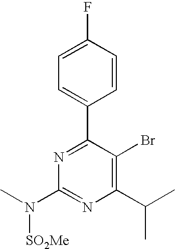

Sodium hydride (1.20 g, 30.0 mmol, 60% suspension in mineral oil) was washed with hexane (2×10 mL), and DMF (50 mL) was then added, followed by 5-bromo-2-chloro-4-(4-fluorophenyl)-6-isopropylpyrimidine (4.944 g, 15.0 mmol). The resulting suspension was cooled to −7° C. and N-methylmethanesulfonamide (2.585 g, 22.5 mmol) was added, washed in with DMF (10 mL). The mixture was stirred for 17.5 hours, then diluted with ethyl acetate (80 mL), toluene (100 mL), and water (120 mL). The organic phase was separated, and the aqueous phase was extracted with a mixture of ethyl acetate (20 mL) and toluene (30 mL). The organic phases were combined, washed with water (2×40 mL) and then brine (20 mL), and dried over anhydrous magnesium sulphate. The solution was concentrated in vacuo (with 2×20 mL hexane azeotropes) to yield the title compound (5.50 g, 91% yield).

¹H NMR (400 MHz; CDCl₃) δ: 1.32 (6H, d), 3.49 (3H, s), 3.55 (3H, s), 3.63 (1H, m), 7.16 (2H, dd), 7.77 (2H, dd). Mp: 122-125° C. HRMS calculated for C₁₃H₁₇N₃O₂FSBr 401.0209, found 401.0225; used in subsequent reaction without further purification.

### 1i): trans-N-(5-(2-Cyanovinyl)-4-(4-fluorophenyl)-6-isopropylpyrimidin-2-yl)-N-methylmethanesulfonamide

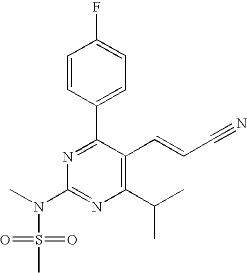

N-(5-Bromo-4-(4-fluorophenyl)-6-isopropylpyrimidin-2-yl)-N-methylmethanesulfonamide (20.0 g, 49.72 mmol), tetra-N-butylammonium bromide (3.24 g, 10 mmol), and bis(tri-tert-butylphosphine)palladium(0) (1.48 g, 2.89 mmol) were charged to a 500 ml round bottom flask. The flask was flushed for five minutes with nitrogen, then toluene (200 mL), dicyclohexylmethylamine (31.6 mL, 147 mmol), acrylonitrile (3.60 mL, 54.67 mmol) were added via syringe and the reaction was stirred. The resulting amber solution was heated in an oil bath at 50° C. for 7 hours, over which time a beige precipitate began to form. The reaction was allowed to cool to room temperature, was diluted with iso-hexane (200 mL), then cooled further to −8° C. The precipitate was collected by filtration and washed with iso-hexane (4×100 mL) to give a crude product (31 g wet) consisting of roughly 85% trans isomer. To the crude product was added methanol (130 mL) and the resulting suspension was stirred at room temperature for 30 minutes, then cooled to −8° C. The white crystalline solids were collected by filtration and dried overnight in a vacuum oven to give the title compound (13.1 g, 70% yield) as a white crystalline solid.

¹H NMR (400 MHz; CDCl₃) δ: 1.32 (6H, d), 3.29 (1H, m), 3.51 (3H, s), 3.58 (3H, s), 5.31 (1H, d), 7.18 (2H, dd), 7.49 (1H, d), 7.58 (2H, dd); Mp: 134.5° C.

HRMS calculated for C₁₈H₁₉FN₄O₂S 374.1213, found 374.1210.

### 1j): trans-N-(4-(4-Fluorophenyl)-6-isopropyl-5-(3-oxoprop-1-enyl)pyrimidin-2-yl)-N-methylmethanesulfonamide

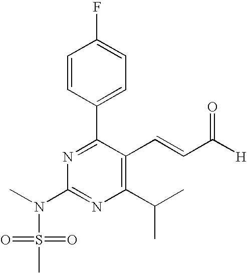

trans-N-(5-(2-Cyanovinyl)-4-(4-fluorophenyl)-6-isopropylpyrimidin-2-yl)-N-methylmethanesulfonamide (12.83 g, 34.27 mmol) was dissolved in toluene (750 mL) and cooled to −9° C. To this solution was added DIBAL (20% solution in toluene, 34 mL, 41.1 mmol) over 45 minutes via syringe pump, maintaining an internal temperature of below −6° C. After the addition was complete, the reaction was allowed to warm slowly to room temperature overnight and then quenched with methanol (3 mL) followed by 1 M HCl (41.1 mL). The resulting suspension was filtered, and lower aqueous layer of the filtrate was separated. The organic layer of the filtrate was treated with 1 M HCl (100 mL), and the resulting suspension was filtered. The layers were separated and the organic layer was washed with brine (125 mL), saturated aqueous NaHCO₃ (125 mL), and water (125 mL), then treated with MgSO₄ and Novit SX 1 G carbon, filtered, and concentrated in vacuo to give 12 g yellow oil. This was purified by chromatography (Biotage cartridge, 100% DCM) to yield the title compound (9.7 g, 76% yield) as a pale yellow amorphous solid.

¹H NMR (400 MHz; CDCl₃) δ: 1.32 (6H, d), 3.39 (1H, m), 3.53 (3H, s), 3.60 (3H, s), 6.22 (1H, dd), 7.15 (2H, dd), 7.52 (1H, d), 7.59 (2H, dd), 9.61 (1H, d); Mp: 86.5° C.

HRMS calculated for C₁₈H₂₀FN₃O₃S 377.1209, found 377.1196.

### 1k): trans-N-(4-(4-Fluorophenyl)-5-(3-hydroxyprop-1-enyl)-6-isopropylpyrimidin-2-yl)-N-methylmethanesulfonamide

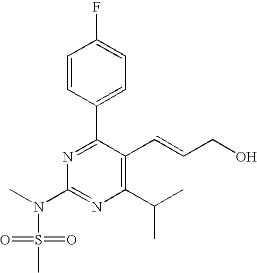

To a room temperature solution of (1,1′-bis(di-tert-butylphosphino)ferrocene)palladium(II) chloride (162 mg, 0.249 mmol) and potassium carbonate (10.3 g, 74.6 mmol) in acetonitrile (40 mL) and water (40 mL) was added trans-4,4,5,5-tetramethyl-2-(3-(tetrahydro-2H-pyran-2-yloxy)prop-1-enyl)-1,3,2-dioxaborolane (see Synthesis, 2004, p. 1814-1820; 11.9 g (70% strength), 31.1 mmol) as a solution in acetonitrile (35 mL) with a water rinse (12.5 mL). The mixture was stirred for 5 minutes, then N-(5-bromo-4-(4-fluorophenyl)-6-isopropylpyrimidin-2-yl)-N-methylmethanesulfonamide (10.0 g, 24.9 mmol) was added as a white solid followed by water (12.5 mL). The reaction was heated to reflux (77° C. internal temperature) for five hours, then allowed to cool to room temperature. It was diluted with MTBE (150 mL) and water (150 mL), separated, and the organic layer was washed twice with water (50 mL) then concentrated in vacuo, providing 16 g of a brown oil. This material was dissolved in 150 mL acetonitrile at room temperature, and 10 M aqueous hydrochloric acid (3.0 mL, 30 mmol) was added. The resulting mixture was stirred for 45 minutes at room temperature, then quenched with sodium bicarbonate (2.52 g, 30 mmol). The mixture was diluted with toluene (150 mL) and water (150 mL), separated, and organic layer was washed twice with water (40 mL). The organic layer was dried over sodium sulfate, concentrated in vacuo, and purified by chromatography (1:1 iso-hexane/EtOAc, 450 g silica gel) to yield the title compound (8.29 g, 72% yield) as a light yellow oil. ¹H NMR (400 MHz) (CDCl₃) δ: 1.27 (6H, d), 3.38 (1H, m), 3.51 (3H, s), 3.57 (3H, s), 4.20 (2H, d), 5.65 (1H, ddd), 6.58 (1H, ddd), 7.09 (2H, dd), 7.59 (2H, dd). HRMS calculated for C₁₈H₂₂FN₃O₃S 379.1366, found 379.1392.

### 1m): trans-N-(4-(4-Fluorophenyl)-6-isopropyl-5-(3-oxoprop-1-enyl)pyrimidin-2-yl)-N-methylmethanesulfonamide

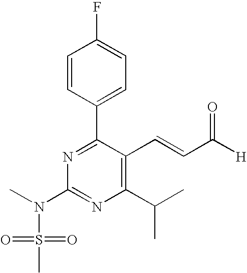

To a room temperature solution of trans-N-(4-(4-fluorophenyl)-5-(3-hydroxyprop-1-enyl)-6-isopropylpyrimidin-2-yl)-N-methylmethanesulfonamide (1.81 g (95% strength), 4.53 mmol) in 25 mL toluene was added manganese dioxide (10 g (85% strength), 97.77 mmol). The resulting suspension was stirred for 18 hours, then filtered through a pad of Celite with a toluene rinse. The solvents were removed from the filtrate in vacuo to give the title compound (1.33 g, 75% yield) as a yellow oil that rapidly became a crystalline solid. ¹H NMR (400 MHz) (CDCl₃) δ: 1.32 (6H, d), 3.39 (1H, m), 3.53 (3H, s), 3.60 (3H, s), 6.22 (1H, dd), 7.15 (2H, dd), 7.52 (1H, d), 7.59 (2H, dd), 9.61 (1H, d). Mp: 86.5° C.

HRMS calculated for C₁₈H₂₀FN₃O₃S 377.1209, found 377.1196.

### Example 2

### (S)-trans-Ethyl 7-(4-(4-fluorophenyl)-6-isopropyl-2-(N-methylmethylsulfonamido)pyrimidin-5-yl)-5-hydroxy-3-oxohept-6-enoate via single-step aldol and hydrolysis

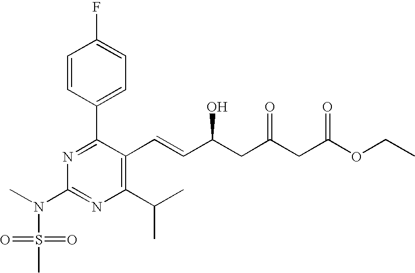

Under a nitrogen atmosphere, trans-N-(4-(4-fluorophenyl)-6-isopropyl-5-(3-oxoprop-1-enyl)pyrimidin-2-yl)-N-methylmethanesulfonamide (200 mg, 0.530 mmol), (S)-(−)-1,1′-bi-(2-naphthyloxy)(diisopropoxy)titanium (48 mg, 0.11 mmol), and lithium chloride (9.0 mg, 0.21 mmol) were dissolved in tetrahydrofuran (5 mL) at room temperature. The resulting red solution was stirred for 5 minutes, then cooled to 0° C. To this solution was added 1,3-diethoxy-1-trimethylsiloxybuta-1,3-diene (244 mg, 1.06 mmol) dropwise over 10 minutes. The resulting mixture was stirred for 42 hours at room temperature, then quenched at 0° C. with 2.0 M aqueous hydrochloric acid (0.75 mL, 1.50 mmol). The resulting solution was warmed to room temperature, stirred for 120 minutes, then diluted with MTBE (20 mL) and water (10 mL). The layers were separated and organic layer washed with water (10 mL), dried with MgSO₄, and concentrated in vacuo to give the crude product. Purification by flash chromatography (Biotage on silica, 2:1 hexane/EtOAc) provided (S)-trans-ethyl 7-(4-(4-fluorophenyl)-6-isopropyl-2-(N-methylmethylsulfonamido)pyrimidin-5-yl)-5-hydroxy-3-oxohept-6-enoate (107.9 mg, 40% yield) as a pale oil in 98.2% enantiomeric excess. ¹H NMR (400 MHz) (CDCl₃) δ: 1.26 (6H, d), 1.28 (3H, t), 2.65 (1H, d), 2.66 (1H, s), 2.89 (1H, br. s), 3.34 (1H, m), 3.44 (2H, s), 3.51 (3H, s), 3.57 (3H, s), 4.21 (2H, q), 4.65 (1H, m), 5.45 (1H, dd), 6.67 (1H, dd), 7.11 (2H, dd), 7.63 (2H, dd). HRMS calculated for C₂₄H₃₀FN₃O₆S 507.1839, found 507.1870.

1. A process for the manufacture of a compound of formula (V) 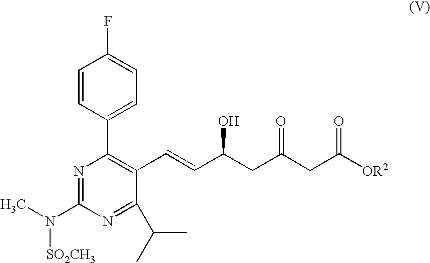
comprising
a) reacting a compound of formula (II) 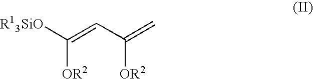
wherein
each R¹ is independently selected from (1-6C)alkyl and phenyl;
each R² is independently selected from (1-6C)alkyl and aryl(1-6C)alkyl, or the two R² groups together comprise a (1-3C)alkylene chain or (5-6C)spirocycloalkyl group optionally substituted with 1 or 2 (1-4C)alkyl groups;
with a compound of formula (III) 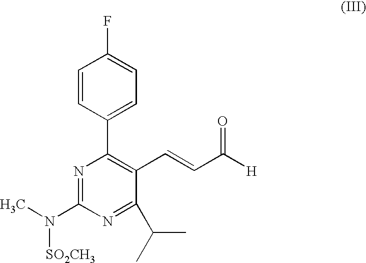
in the presence of a titanium (IV) catalyst of formula (IV) 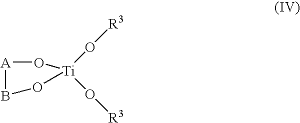
and an alkali metal halide salt, in an inert solvent,
wherein
each R³ is independently selected from (1-6C)alkyl and
A-B comprises an optionally substituted biaryl derivative in the S-configuration.

2. The process for the manufacture of a compound of formula (V) as claimed in claim 1
comprising reacting a compound of formula (II) with a compound of formula (III), in the presence of a titanium(IV) catalyst of formula (IV) and an alkali metal halide salt in an inert solvent to give a compound of formula (Va); 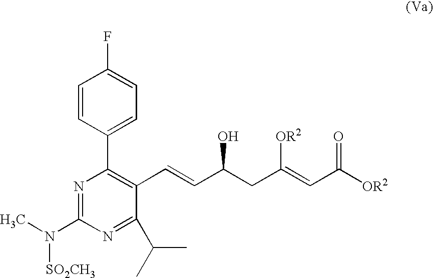
and hydrolyzing (Va) to give a compound of formula (V).

3. A process for the manufacture of a compound of formula (VI) comprising
a) forming a compound of formula (V) according to claim 1; and
b) reducing the keto-group in the compound of formula (V) to give a compound of formula (VI)

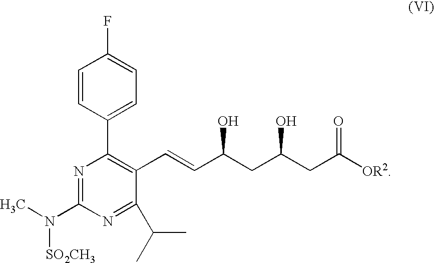

4. A process for forming a compound of formula (I) or a pharmaceutically acceptable salt thereof, comprising
a) forming a compound of formula (V);
b) forming a compound of formula (VI) according to claim 3;
c) removing the R² group to give the compound of formula (I) or a salt thereof; and
optionally forming a pharmaceutically-acceptable salt of the compound of formula (I)

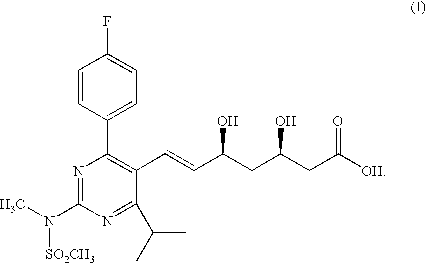

5. The process according to claim 4 wherein steps b) and c) are carried out without isolation of the intermediate compound of formula (VI).

6. The process according to claim 1 wherein the alkali metal halide is lithium chloride.

7. The process according to claim 1 wherein each R¹ is methyl.

8. The process according to claim 1 wherein each R² is independently selected from (1-6C)alkyl.

9. The process according to claim 8 wherein each R² is ethyl.

10. The process according to any one of claims 4 to 9 wherein the compound of formula (I) is isolated as its calcium salt.

11. The process according to claim 1 wherein the compound of formula (IV) is (S)-(−)-1,1′-bi-(2-naphthyloxy)(diisopropoxy)titanium:

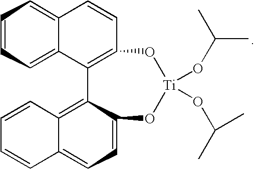

12. The compound (S)-trans-ethyl 3-ethoxy-7-(4-(4-fluorophenyl)-6-isopropyl-2-(N-methylmethylsulfonamido)pyrimidin-5-yl)-5-hydroxyhept-2,6-dienoate [chemical structure]
